(19) United States
(12) Patent Application Publication
(10) Pub. No.: US 2012/0076813 A1
(43) Pub. Date: Mar. 29, 2012

(54) USES OF GLUTAMYL tRNA SYNTHETASE (GtS) FRAGMENTS

(75) Inventors: Yaffa Mizrachi Nebenzahl, Beer Sheva (IL); Ron Dagan, Omer (IL)

(73) Assignee: BEN GURION UNIVERSITY OF THE NEGEV RESEARCH AND DE VELOPMENT AUTHORITY, Beer Sheva (IL)

(21) Appl. No.: 13/132,459

(22) PCT Filed: Nov. 30, 2009
(86) PCT No.: PCT/IL2009/001122

§ 371 Date: Aug. 23, 2011

Related U.S. Application Data
(60) Provisional application No. 61/119,422, filed on Dec. 3, 2008.

(51) Int. Cl. A61K 39/09; A61P 37/04; A61P 11/00; A61P 31/04

(52) U.S. Cl. 424/190.1

(57) ABSTRACT

The present invention relates to polypeptide fragments, including variants and analogs, of Streptococcus pneumonia (S. pneumoniae) glutamyl tRNA synthetase (GtS) protein and compositions comprising the same for use in preventing adhesion of S. pneumoniae to respiratory tract cells, cell-to-cell spread and bacteremia. In particular, the present invention relates to the use of such polypeptides for treating S. pneumoniae infection.

### FIELD OF THE INVENTION

The present invention relates to the protein glutamyl tRNA synthetase (GtS) derived from Streptococcus pneumonia (S. pneumoniae) cell wall. In particular, the present invention relates to their use as a treatment against S. pneumoniae infection.

### BACKGROUND OF THE INVENTION

Streptococcus pneumoniae belongs to the commensal flora of the human respiratory tract, but can also cause invasive infections such as meningitis and sepsis. Mortality due to pneumococcal infection remains high all over the world, augmented by a wide-spread antibiotic resistance in many pneumococcal strains (Dagan et al., Pneumococcal Infections. In: Feigin R, et al, ed. Textbook of Pediatric Infectious Diseases. 5 ed. Philadelphia: Saunders Co, 2004:1204-58). The current polysaccharide based vaccines (including polysaccharide conjugates), elicit a strain specific protection in children and the elderly, who are the main targets for pneumococcal infections. However the available vaccines either do not elicit long lasting protection or are limited in strain coverage. Development of new preventive and therapeutic interventions is hampered due to the incomplete understanding of pneumococcal pathogenesis.

Most children in the developing world become nasopharyngeal carriers of Streptococcus pneumoniae. Many develop pneumococcal disease that can be invasive (such as bacteremia, sepsis or meningitis), or mucosal infections (such as pneumonia and otitis media). S. pneumoniae is the leading cause of non-epidemic childhood meningitis in Africa and other regions of the developing world. Approximately, one to two million children die from pneumococcal pneumonia each year. Specifically, when considering deaths of children under five years old worldwide, about 20% is from pneumococcal pneumonia. These high morbidity and mortality rates and the persistent emergence of antibiotic resistant strains of S. pneumoniae heighten the need to develop an effective means of prevention, such as vaccination. The optimal anti-pneumococcal vaccine should be safe, efficacious, wide-spectrum (covering most pneumococcal strains) and affordable (cheap and available in large quantities). The existing pneumococcal polysaccharide and polysaccharide-conjugated vaccines protect against a narrow but significant group of pneumococcal serotypes, vaccinated subjects remaining susceptible to strains not covered by the vaccines. Of note, the current pneumococcal conjugate vaccines generally have lower coverage against pneumococcal strains causing disease in the developing world compared to developed countries. In addition to limitations of coverage, conjugate vaccines are complex to produce and expensive, resulting in restricted quantities and are beyond the budget of many poor countries.

The search for wide range anti-pneumococcal vaccine is ongoing. Indeed, introduction of pneumococcal 7-valent polysaccharide conjugate vaccine reduced significantly the rates of invasive diseases in infants and restricted significantly the rates of invasive diseases in the non vaccinated members of the community (Kyaw et al., N. Engl. J. Med. 2006, 354, 1455-63). However, carriage and diseases resulting from strains not included in the vaccine are on the rise (blusher D M., N. Engl. J. Med. 2006, 354, 1522-4, Huang et al., Pediatrics 2005, 116, e408-13).

The mucosal epithelial surfaces with their tight junctions constitute the first line of defense that prevents the entry of pathogens and their products. S. pneumoniae adhere to the nasopharyngeal mucosal cells (Tuomanen E. 1999 Curr. Opin. Microbiol., 2:35-9) causing carriage without an overt inflammatory response. For clinical disease to occur, S. pneumoniae have to spread from the nasopharynx into the middle ear or the lungs or cross the mucosal epithelial cell layer and be deposited basally within the submucosa (Ring et al., J. Clin. Invest. 1998, 102:347-60). Molecules involved in adhesion, spread and invasion of S. pneumoniae, include capsular polysaccharides, cell-wall peptidoglycan and surface proteins (Jedrzejas M J. Microbiol. Mol. Biol. Rev. 2001, 65, 187-207).

It has been observed that in infants the antibody response to S. pneumoniae increases with age and correlates negatively with morbidity (Lifshitz et al. Clin. Exp. Immunol. 2002, 127, 344-53). To identify these proteins a longitudinal series of children's sera was utilized to survey which S. pneumoniae cell wall associated proteins exhibit age-dependent antigenicity together with biochemical and proteomic studies, (Ling et al., Clin Exp Immunol 2004, 138, 290-8). One such protein is Glutamyl tRNA Synthetase (GtS).

Mizrachi-Nebenzahl et al. 2007 (J. Infect. Dis., 196, 945-53) discloses that Streptococcus pneumoniae surface-exposed intact GtS, is able to induce a partially protective immune response in mice.

International Patent Application Publication No. WO 02/077021, assigned to Chiron S.P.A., discloses the sequence of about 2,500 S. pneumoniae genes, including the GtS gene, and their corresponding amino acid sequences from S. pneumoniae type 4 strain that were identified in silico. The use of a subset of 432 of those sequences as antigens for immunization is also suggested although no working examples for the use of the proteins as antigens in the production of vaccines are provided.

International Patent Application Publication No. WO 97/38718 assigned to SmithKline Beecham Corp. discloses S. pneumoniae GtS polypeptides of 480, 348, 126 and 62 amino acids, polynucleotides encoding the GtS polypeptides and methods for producing such polypeptides by recombinant techniques. Also provided are vaccine formulations comprising GtS polypeptides although no such vaccine was actually prepared at the time of filing. U.S. Pat. No. 5,958,734 claims GtS N-terminus fragment of 348 and C-terminus 126 amino acids fragment. U.S. Pat. No. 5,976,840 claims a 480 amino acids GtS sequence starting at Val-7, and variants containing up to three nucleotide substitutions, deletions, or nucleotide insertions for every 100 nucleotides. U.S. Pat. No. 6,300,119 claims a GtS variant polynucleotide comprising a sequence identical to the polynucleotide encoding the above 480 amino acids polypeptide, except that up to five nucleotides may be substituted, deleted or inserted for every 100 nucleotides; and wherein the first polynucleotide sequence detects Streptococcus pneumoniae by hybridization. U.S. Pat. No. 6,165,760 relates to the GtS polypeptide the above of 480 amino acids sequence further comprising a heterologous amino acid sequence.

WO 03/082183 to one of the inventors of the present application discloses a defined group of cell wall and cell membrane S. pneumoniae proteins for use as vaccines against said bacteria. The thirty eight identified S. pneumoniae proteins, including the intact GtS, were found to be immunogenic in age groups which do not produce S. pneumoniae antibodies following inoculation with polysaccharide-based vaccines.

### SUMMARY OF THE INVENTION

In one embodiment, this invention provides a method of inhibiting adhesion of S. pneumoniae to cells of the respiratory tract, said method comprising contacting cells of a respiratory tract with a synthetic or recombinant polypeptide of 50-250 amino acids derived from the sequence of Streptococcus pneumonia (S. pneumoniae) glutamyl tRNA synthetase (GtS) of SEQ ID NO:1, comprising the sequence KNADLETIFEMAKPFLEEAGRLTDKAEKL (SEQ ID NO:2), and variants and analogs thereof, wherein said synthetic or recombinant polypeptide prevents adhesion of S. pneumoniae to cells of said respiratory tract.

In one embodiment, the invention provides a method of treating a subject infected with S. pneumoniae, said method comprising administering to said subject a synthetic or recombinant polypeptide of 50-250 amino acids derived from the sequence of Streptococcus pneumonia (S. pneumoniae) glutamyl tRNA synthetase (GtS) of SEQ ID NO:1, comprising the sequence KNADLETIFEMAKPFLEEAGRLTDKAEKL (SEQ ID NO:2), and variants and analogs thereof. According to this aspect, and in one embodiment, the subject is afflicted with sepsis. In some embodiments, the subject is predisposed to developing sepsis.

In a first aspect the present invention provides a synthetic or recombinant polypeptide of 50-250 amino acids derived from the sequence of S. pneumoniae GtS (SEQ ID NO:1), comprising the sequence KNADLETIFEMAKPFLEEAGRLTDKAEKL (SEQ ID NO:2), and variants and analogs thereof.

Variants include substitution of one amino acid residue per each ten amino acid residues in a polypeptide sequence, namely, polypeptides having 90% or more identity are included within the scope of the present invention. According to some preferred embodiments, sequences having at least 97% identity to the polypeptides of the present invention are provided.

According to some preferred embodiments the polypeptide consists of 100-200 amino acids.

According to some embodiments, the GtS polypeptide according to the invention share less than 30% identity with the human GtS protein of Human GtS, GENE ID: 124454 EARS2. According to other embodiments, the GtS polypeptide according to the invention share less than 10% identity with the human GtS protein of Human GtS, GENE ID: 124454 EARS2. According to yet another embodiment, when aligning the sequence of a GtS polypeptide according to the invention with Human GtS, GENE ID: 124454 EARS2, no more than six contiguous amino acid residues are identical between the two sequences.

According to some embodiments the present invention provides a synthetic or recombinant GtS polypeptide fragment comprising the sequence:

XKNADLETIFEMAKPFLEEAGRLTDKAEKLVELYKPQMKSVDEIIPLTDLFFSDFPEL TEAEREVMTGETVPTVLEAFKAKLEAMTDDKFVTENIFPQIKAVQKETGIKGKNLFM PIRIAVSGEMHGPELPDTIFLLGREKSIQHIENMLKEISK (SEQ ID NO:3, residues 333-486 of SEQ ID NO:1), wherein X is Methionine or represents the polypeptide's N-terminus, and variants and analogs thereof.

According to other embodiments the synthetic or recombinant GtS polypeptide fragment comprises the sequence:
XKNADLETIFEMAKPFLEEAGRLTDKAEKLVELYKPQMKSVDEIIPLTDX₁ FFSDFPELTEAEREVMTX₂ETVPTVLEAFKAKLEAMTDDX₃FVTENIFPQIK AVQKETGIKGKNLFMPIRIAVSGEMHGPELPDTX₄FLLGREKSIQHIENX₅L KEISK (SEQ ID NO:4), wherein X is Methionine or represents the polypeptide's N-terminus, X₁ is L or F, X₂ is G or D, X₃ is K or E, X₄ is I or V, and X₅ is M or I, and variants and analogs thereof.

According to yet other embodiments the synthetic or recombinant GtS polypeptide fragment comprises a sequence selected from the group consisting of:
XKNADLETIFEMAKPFLEEAGRLTDKAEKLVELYKP (SEQ ID NO:5);
KNADLETIFEMAKPFLEEAGRLTDKAEKLVELYKPQMKSVDEIIPLIDLFFSD FP (SEQ ID NO:6);
XKNADLETIFEMAKPFLEEAGRLTDKAEKLVELYKPQMKSVDEIIPLTDLFFS DFPELTEAEREVMTGETVPTVLEAFKAK (SEQ ID NO:7);
XKNADLETIFEMAKPFLEEAGRLTDKAEKLVELYKPQMKSVDEIIPLTDLFFS DFPELTEAEREVMTGETVPTVLEAFKAKLEAMTDDKFVTENIFPQIKAVQKE T (SEQ ID NO:8);
XKNADLETIFEMAKPFLEEAGRLTDKAEKLVELYKPQMKSVDEIIPLTDLFFS DFPELTEAEREVMTGETVPTVLEAFKAKLEAMTDDKFVTENIFPQIKAVQKE TGIKGKNLFMPIRIAVSG (SEQ ID NO:9); and
XKNADLETIFEMAKPFLEEAGRLTDKAEKLVELYKPQMKSVDEIIPLTDLFFS DFPELTEAEREVMTGETVPTVLEAFKAKLEAMTDDKFVTENIFPQIKAVQKE TGIKGKNLFMPIRIAVSGEMHGPELPDTIFLLGR (SEQ ID NO:10), wherein X is Methionine or represents the polypeptide's N-terminus, and variants and analogs thereof.

According to yet other embodiments the present invention provides a synthetic or recombinant GtS polypeptide fragment consisting of a sequence selected from the group of SEQ ID NO:3 to SEQ ID NO:10.

According to some embodiments the polypeptide fragments are not conjugated or fused to a carrier protein. In other embodiments the polypeptide fragments of the present invention are produced as a recombinant fusion protein comprising a carrier sequence, namely the fragments are inserted within a sequence of a carrier polypeptide or are fused to an amino terminal or a carboxy terminal portion of a carrier protein sequence (or other S. pneumoniae proteins or polypeptide).

The present invention provides, according to another aspect, isolated polynucleotide sequences encoding the GtS fragment polypeptides.

According to some embodiments the isolated polynucleotide sequences encode a polypeptide sequence of 50-250 amino acids comprising the sequence KNADLETIFEMAKPFLEEAGRLTDKAEKL (SEQ ID NO:2), and variants and analogs thereof. According to some preferred embodiments the isolated polynucleotide sequences encode a polypeptide sequence consisting of 100-200 amino acids.

According to some specific embodiments the isolated polynucleotide sequence comprises SEQ ID NO:11. According to some specific embodiments the isolated polynucleotide sequence consists of SEQ ID NO:11.

According to additional embodiments the isolated polynucleotide sequence encode a polypeptide sequence selected from the group consisting of: SEQ ID NO:3 to SEQ ID NO:10, and variants and analogs thereof.

According to yet another aspect, the present invention provides compositions comprising at least one synthetic or recombinant GtS polypeptide fragment of 50-250 amino acids comprising the sequence KNADLETIFEMAKPFLEEAGRLTDKAEKL (SEQ ID NO:2), and variants and analogs thereof. According to some preferred embodiments the polypeptide consists of 100-200 amino acids.

According to some embodiments the composition comprises a GtS polypeptide sequence selected from the group consisting of: SEQ ID NO:3 to SEQ ID NO:10, and variants and analogs thereof.

According to other embodiments, a composition according to the present invention further comprises at least one additional S. pneumoniae polypeptide or protein sequence.

In some embodiments, the invention provides for the use of a polypeptide according to the invention for preparation of a composition against S. pneumoniae, as well as use of an isolated polynucleotide according to the invention for production of a GtS polypeptide fragment of 50-250 amino acids comprising the sequence KNADLETIFEMAKPFLEEAGRLTDKAEKL (SEQ ID NO:2), and variants and analogs thereof. According to some preferred embodiments the polypeptide consists of 100-200 amino acids.

In some embodiments, the invention provides for the use of a synthetic or recombinant polypeptide of 50-250 amino acids derived from the sequence of Streptococcus pneumonia (S. pneumoniae) glutamyl tRNA synthetase (GtS) of SEQ ID NO:1, comprising the sequence KNADLETIFEMAKPFLEEAGRLTDKAEKL (SEQ ID NO:2), and variants and analogs thereof in the preparation of a medicament for use in inhibiting adhesion of S. pneumoniae to cells of the respiratory tract in a subject.

In some embodiments, the invention provides for the use of a synthetic or recombinant polypeptide of 50-250 amino acids derived from the sequence of Streptococcus pneumonia (S. pneumoniae) glutamyl tRNA synthetase (GtS) of SEQ ID NO:1, comprising the sequence KNADLETIFEMAKPFLEEAGRLTDKAEKL (SEQ ID NO:2), and variants and analogs thereof in the preparation of a medicament for use in treating a subject infected with S. pneumoniae.

All the polypeptides disclosed in the present invention can be produced by recombinant methods and by chemical synthesis.

Another aspect of the present invention provides a fusion protein comprising at least one GtS fragment polypeptide and at least one additional polypeptide.

According to one embodiment the fusion protein comprises a GtS polypeptide fragment of 100-200 amino acids comprising the sequence KNADLETIFEMAKPFLEEAGRLTDKAEKL (SEQ ID NO:2), and variants and analogs thereof.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 shows PCR amplification by genomic DNA of the GtS fragment (333-486).

FIG. 2 depicts a gel confirming of the existence of the expected 462 bp insert by PCR amplification.

FIG. 3 represents resolution of the eluted GtS fragment 333-486 (23 kDa band) by 1D-PAGE stained with Coomassie Brilliant Blue.

FIG. 4 shows western blot analysis of the recombinant GtS fragment 333-486 HIS-tagged fusion protein (23 kDa band) by 1D-PAGE using anti-HIS-tagged antibodies.

FIG. 5 demonstrates the inhibition of S. pneumoniae adhesion to A549 lung derived epithelial cells by the recombinant GtS fragment. A. Inhibition of unencapsulated serotype 2 strain R6 adhesion. B. Inhibition of serotype 2 strain D39 adhesion. C. Inhibition of unencapsulated serotype 3 strain 3.8 adhesion D. Inhibition of serotype 3 strain WU2 adhesion E. Inhibition of unencapsulated serotype 14 strain 14.8 adhesion F. Inhibition of serotype 14 strain 14 adhesion

### DETAILED DESCRIPTION OF THE INVENTION

The present invention provides polypeptides derived from the sequence of S. pneumoniae GtS protein. A polypeptide according to the present invention comprises the 29 amino acid residues KNADLETIFEMAKPFLEEAGRLTDKAEKL (SEQ ID NO:2) corresponding to residues 333-361 of the intact S. pneumoniae GtS protein of SEQ ID NO:1.

A polypeptide fragment of 154 amino acids corresponding to amino acid residues 333-486 of the S. pneumoniae GtS protein was produced, characterized and found to be effective in producing neutralizing antibodies in rabbits against S. pneumoniae infection.

For convenience, certain terms employed in the specification, examples and claims are described herein.

“Amino acid sequence”, as used herein, refers to an oligopeptide, peptide, polypeptide, or protein sequence, and fragment thereof, and to naturally occurring or synthetic molecules.

A “chimeric protein” or “fusion protein” are used interchangeably and refer to a polypeptide operatively linked to a polypeptide other than the polypeptide from which the GtS polypeptide fragment was derived.

### Recombinant Production of Polypeptides

The polypeptide fragments of the present invention can be prepared by expression in an expression vector per se or as a chimeric protein. The methods to produce a chimeric or recombinant protein comprising one or more GtS polypeptide fragment are known to those with skill in the art. A nucleic acid sequence encoding one or more GtS polypeptide fragment can be inserted into an expression vector for preparation of a polynucleotide construct for propagation and expression in host cells.

The term “expression vector” and “recombinant expression vector” as used herein refers to a DNA molecule, for example a plasmid or virus, containing a desired and appropriate nucleic acid sequences necessary for the expression of the recombinant polypeptides for expression in a particular host cell. As used herein “operably linked” refers to a functional linkage of at least two sequences. Operably linked includes linkage between a promoter and a second sequence, for example an nucleic acid of the present invention, wherein the promoter sequence initiates and mediates transcription of the DNA sequence corresponding to the second sequence.

The regulatory regions necessary for transcription of the polypeptides can be provided by the expression vector. The precise nature of the regulatory regions needed for gene expression may vary among vectors and host cells. Generally, a promoter is required which is capable of binding RNA polymerase and promoting the transcription of an operably-associated nucleic acid sequence. Regulatory regions may include those 5′ non-coding sequences involved with initiation of transcription and translation, such as the TATA box, capping sequence, CAAT sequence, and the like. The non-coding region 3′ to the coding sequence may contain transcriptional termination regulatory sequences, such as terminators and polyadenylation sites. A translation initiation codon (ATG) may also be provided.

In order to clone the nucleic acid sequences into the cloning site of a vector, linkers or adapters providing the appropriate compatible restriction sites are added during synthesis of the nucleic acids. For example, a desired restriction enzyme site can be introduced into a fragment of DNA by amplification of the DNA by use of PCR with primers containing the desired restriction enzyme site.

An expression construct comprising a GtS polypeptide fragment sequence operably associated with regulatory regions can be directly introduced into appropriate host cells for expression and production of polypeptide per se or as recombinant fusion protein. The expression vectors that may be used include but are not limited to plasmids, cosmids, phage, phagemids or modified viruses. Typically, such expression vectors comprise a functional origin of replication for propagation of the vector in an appropriate host cell, one or more restriction endonuclease sites for insertion of the desired gene sequence, and one or more selection markers.

The recombinant polynucleotide construct comprising the expression vector and a GtS polypeptide fragment should then be transferred into a bacterial host cell where it can replicate and be expressed. This can be accomplished by methods known in the art. The expression vector is used with a compatible prokaryotic or eukaryotic host cell which may be derived from bacteria, yeast, insects, mammals and humans.

Once expressed by the host cell, the GtS polypeptide fragment can be separated from undesired components by a number of protein purification methods. One such method uses a polyhistidine tag on the recombinant protein. A polyhistidine-tag consists in at least six histidine (His) residues added to a recombinant protein, often at the N- or C-terminus. Polyhistidine-tags are often used for affinity purification of polyhistidine-tagged recombinant proteins that are expressed in E. coli or other prokaryotic expression systems. The bacterial cells are harvested by centrifugation and the resulting cell pellet can be lysed by physical means or with detergents or enzymes such as lysozyme. The raw lysate contains at this stage the recombinant protein among several other proteins derived from the bacteria and are incubated with affinity media such as NTA-agarose, HisPur resin or Talon resin. These affinity media contain bound metal ions, either nickel or cobalt to which the polyhistidine-tag binds with micromolar affinity. The resin is then washed with phosphate buffer to remove proteins that do not specifically interact with the cobalt or nickel ion. The washing efficiency can be improved by the addition of 20 mM imidazole and proteins are then usually eluted with 150-300 mM imidazole. The polyhistidine tag may be subsequently removed using restriction enzymes, endoproteases or exoproteases. Kits for the purification of histidine-tagged proteins can be purchased for example from Qiagen.

Another method is through the production of inclusion bodies, which are inactive aggregates of protein that may form when a recombinant polypeptide is expressed in a prokaryote. While the cDNA may properly code for a translatable mRNA, the protein that results may not fold correctly, or the hydrophobicity of the sequence may cause the recombinant polypeptide to become insoluble. Inclusion bodies are easily purified by methods well known in the art. Various procedures for the purification of inclusion bodies are known in the art. In some embodiments the inclusion bodies are recovered from bacterial lysates by centrifugation and are washed with detergents and chelating agents to remove as much bacterial protein as possible from the aggregated recombinant protein. To obtain soluble protein, the washed inclusion bodies are dissolved in denaturing agents and the released protein is then refolded by gradual removal of the denaturing reagents by dilution or dialysis (as described for example in Molecular cloning: a laboratory manual, 3rd edition, Sambrook, J. and Russell, D. W., 2001; CSHL Press).

### Formulation

The formulation can contain a variety of additives, such as excipient, stabilizers, buffers, or preservatives.

In preferred embodiment, the administration is oral.

According to one embodiment of the invention, the formulation may be applied to the lymphatic tissue of the nose in any convenient manner. However, it is preferred to apply it as a liquid stream or liquid droplets to the walls of the nasal passage. The intranasal composition can be formulated, for example, in liquid form as nose drops, spray, or suitable for inhalation, as powder, as cream, or as emulsion.

In another embodiment of the invention, administration is oral, for example, in the form of a tablet or encased in a gelatin capsule or a microcapsule.

The formulation of these modalities is general knowledge to those with skill in the art.

Liposomes provide another delivery system. Liposomes are bilayered vesicles composed of phospholipids and other sterols surrounding a typically aqueous center where products can be encapsulated. The liposome structure is highly versatile with many types range in nanometer to micrometer sizes, from about 25 nm to about 500 μm. Liposomes have been found to be effective in delivering therapeutic agents to mucosal surfaces. The average survival time or half life of the intact liposome structure can be extended with the inclusion of certain polymers, for example polyethylene glycol, allowing for prolonged release in vivo. Liposomes may be unilamellar or multilamellar.

In some embodiments, the liposome further comprises a targeting moiety, which specifically targets a desired organ or tissue or cell type, as will be appreciated by the skilled artisan. In some embodiments, the targeting moiety may be any such appropriate moiety, including an antibody, a lectin, a peptide, or others, as known to the skilled artisan. Similarly, the liposome may further comprise a second therapeutic agent, as further described hereinbelow.

It is to be understood that any composition as herein described may be useful in the treatment of S. pneumoniae infection. Similarly, it is to be understood that any composition as herein described may be useful in the prevention of S. pneumoniae adhesion to cells of the respiratory tree underlying vasculature, in the lung mucosa or lung parenchyma. In some embodiments, the compositions as herein described may be useful in the treatment of extra-pulmonary seeding by S. pneumoniae, including seeding of draining lymph nodes.

In some applications an excipient may be included in the formulation. A preferred mode of administration is intranasal administration.

Compositions comprising different GtS fragments can be produced by mixing or linking a number of different GtS polypeptide fragments according to the invention. In addition, GtS fragments according to the present invention may be included in a composition comprising any other S. pneumoniae protein or protein fragment, including mutated proteins such as detoxified pneumolysin, or they can be linked to or produced in conjunction with any such S. pneumoniae protein or protein fragment.

In one embodiment the compositions of this invention comprise a polypeptide of this invention, alone or in some embodiments, in combination with a second pharmaceutically active or therapeutic agent. In one embodiment, the term “pharmaceutically active agent” refers to any medicament which satisfies the indicated purpose. In some embodiments, the term “agent” of this invention is a decongestant, antibiotic, bronchodilator, anti-inflammatory steroid, leukotriene antagonist or histamine receptor antagonist, and the like.

In some embodiments, the polypeptides of this invention comprise a fusion protein, wherein a second therapeutic agent is fused to the polypeptides of this invention, or in some embodiments, a targeting moiety is fused to a polypeptide of this invention. In some embodiments, such fusion proteins may further comprise an appropriate spacer, and an internal cleavage site, for cleavage between the polypeptide of the invention and the second therapeutic target and/or targeting moiety.

In one embodiment, decongestants are medicines used to relieve nasal congestion caused by swelling of the membranes lining the nasal passages. Decongestants relieve the swelling by reducing the blood supply to the swollen membranes, causing the membranes to shrink. Although any suitable decongestant can be used, the preferred decongestants of the present invention are pseudoephedrine, a pharmaceutically acceptable pseudoephedrine salt, and mixtures thereof, as well as a phenylephrine salt. Pseudoephedrine is a sympathomimetic amine. Any suitable pseudoephedrine salt may be used in the present invention; however, pseudoephedrine hydrochloride, (+)-pseudoephedrine sulfate, and/or phenylephrine salt such as phenylephrine hydrochloride, are typically used. Other suitable pseudoephedrine salts include sodium, hydrofluoric, sulfuric, sulfonic, tartic, fumaric, hydrobromic, glycolic, citric, maleic, phosphoric, succinic, acetic, nitric, benzoic, ascorbic, p-toluene, benzenesulfonic, naphthalenesulfonic, propionic, and the like. In addition to pseudoephedrine, other suitable decongestants include oxymetazoline, phenylpropanolamine, and other sympathomimetic drugs.

In one embodiment, examples of leukotriene antagonist agents are montelukast and zafirlukast which block the actions of cysteinyl leukotrienes at the CysLT1 receptor on target cells such as bronchial smooth muscle.

In one embodiment, examples of bronchodilators are metaproterenol, isoetherine, terbutaline, albuterol and atropine sulfate.

In one embodiment, examples of histamine receptor antagonist are loratadine or desloratadine. Other antihistamines that may be utilized include H1 antagonist antihistamines including: ethylenediamines, such as mepyramine (pyrilamine) and antazoline; ethanolamines, such as diphenhydramine, carbinoxamine, doxylamine, clemastine, dimenhydrinate; alkylamines, such as pheniramine, chlorphenamine (chlorpheniramine), dexchlorphenamine, brompheniramine, triprolidine; piperazines, such as hydroxyzine and meclizine; tricyclics, such as promethazine, alimemazine (trimeprazine), cyproheptadine, azatadine; acrivastine; astemizole; cetirizine, levocetirizine, fexofenadine, loratadine, desloratadine, mizolastine, and terfenadine.

In one embodiment, the medicament is an anti-infective agent. In one embodiment, the anti-infective agent is an antibiotic agent. In one embodiment, the antibiotic agent is a beta-lactam antibiotic. In one embodiment, beta-lactam antibiotics include, but are not limited to, penicillin, benzathine penicillin, benzylpenicillin, amoxicillin, procaine penicillin, dicloxacillin, amoxicillin, flucloxacillin, ampicillin, methicillin, azlocillin, carbenicillin, ticarcillin, mezlocillin, piperacillin, phenoxymethylpenicillin, co-amoxiclav, cephalosporin, cefalexin, cephalothin, cefazolin, cefaclor, cefuroxime, cefamandole, cefotetan, cefoxitin, ceftriaxone, cefotaxime, ceftazidime, cefepime, cefpirome, imipenem, meropenem, ertapenem, faropenem, monobactam, aztreonam, or carbapenem.

In one embodiment, the antibiotic is a tetracycline antibiotic. In one embodiment, tetracycline antibiotics include, but are not limited to, tetracycline, chlortetracycline, demeclocycline, doxycycline, lymecycline, minocycline, or oxytetracycline.

In one embodiment, the antibiotic is a macrolide antibiotic. In one embodiment, macrolide antibiotics include, but are not limited to, erythromycin, azithromycin, oxithromycin, dirithromycin, clarithromycin, josamycin, oleandomycin, kitasamycin, spiramycin, tylosin/tylocine, troleandomycin, carbomycin, cethromycin, or telithromycin.

In one embodiment, the antibiotic is an aminoglycoside antibiotic. In one embodiment, aminoglycoside antibiotics include, but are not limited to, gentamicin, tobramycin, faropenem, imipenem, kanamycin, neomycin, ertapenem, apramycin, paromomycin sulfate, streptomycin, or amikacin.

In one embodiment, the antibiotic is a quinolone antibiotic. In one embodiment, quinolone antibiotics include, but are not limited to, ciprofloxacin, norfloxacin, lomefloxacin, enoxacin, ofloxacin, ciprofloxacin, levofloxacin, sparfloxacin, gatifloxacin, moxifloxacin, trovafloxacin, or alatrofloxacin.

In one embodiment, the antibiotic is a cyclic peptide antibiotic. In one embodiment, cyclic peptide antibiotics include, but are not limited to, vancomycin, streptogramins, Microcin J25, Bacteriocin AS-48, RTD-1, or polymyxins.

In one embodiment, the antibiotic is a lincosamide antibiotic. In one embodiment, lincosamide antibiotics include, but are not limited to, clindamycin.

In one embodiment, the antibiotic is an oxazolidinone antibiotic. In one embodiment, oxazolidinone antibiotics include, but are not limited to, linezolid, U-100592, DA-7867, AZD2563, or U-100766.

In one embodiment, the antibiotic is a sulfa antibiotic. In one embodiment, sulfa antibiotics include, but are not limited to, sulfisoxazole.

In one embodiment, the antibiotic is an antiseptic agent. In one embodiment, antiseptic agents include, but are not limited to, alcohols, chlorhexidine, chlorine, hexachlorophene, iodophors, chloroxylenol (PCMX), quaternary ammonium compounds, or triclosan.

In one embodiment, the medicament may be a growth factor such as epidermal growth factor (EGF), transforming growth factor-α (TGF-α), platelet derived growth factor (PDGF), fibroblast growth factors (FGFs) including acidic fibroblast growth factor (α-FGF) and basic fibroblast growth factor (β-FGF), transforming growth factor-β (TGF-β) and insulin like growth factors (IGF-1 and IGF-2), or any combination thereof.

In one embodiment, the medicament may be a local anesthetic agent. In one embodiment, local anesthetic agents include, but are not limited to benzocaine, chloroprocaine, cocaine, procaine, bupivacaine, levobupivacaine, lidocaine, mepivacaine, prilocalne, or ropivacaine. In one embodiment, the medicament may be a general anaesthetic agent. In one embodiment, general anesthetic agents include, but are not limited to, esflurane, sevoflurane, isoflurane, halothane, enflurane, methoxyflurane, xenon, propofol, etomidate, methohexital, midazolam, diazepamor, ketamine, thiopentone/thiopental, or lidocaine/prilocalne.

In one embodiment, the medicament may be an analgesic agent. In some embodiments, analgesic agents include, but are not limited to, paracetamol or non-steroidal anti-inflammatory agent. In some embodiments, analgesic agents include opiates or morphinomimetics such as morphine, pethidine, oxycodone, hydrocodone, diamorphine, tramadol, or buprenorphine. In some embodiments, a combination of two or more analgesics is desired.

In one embodiment, the medicament may be a sedative agent. In one embodiment, the sedative agent is an antidepressant agent such as mirtazapine or trazodone. In one embodiment, the sedative agent is a barbiturate such as secobarbital, pentobarbital, or amobarbital. In one embodiment, the sedative agent is a benzodiazepine such as diazepam, clonazepam, alprazolam, temazepam, chlordiazepoxide, flunitrazepam, lorazepam, or clorazepate. In one embodiment, the sedative agent is an imidazopyridines such as zolpidem or alpidem. In one embodiment, the sedative agent is a pyrazolopyrimidine such as zaleplon. In one embodiment, the sedative agent is an antihistamine such as diphenhydramine, dimenhydrinate, or doxylamine. In one embodiment, the sedative agent is an antipsychotic agent such as ziprasidone, risperidone, quetiapine, clozapine, prochlorperazine, perphenazine, loxapine, trifluoperazine, thiothixene, haloperidol, or fluphenazine. In one embodiment, the sedative agent is an herbal sedative such as valerian plant mandrake, or kava. In some embodiments, the sedative agent is eszopiclone, ramelteon, methaqualone, ethchlorvynol, chloral hydrate, meprobamate, glutethimide, methyprylon, gamma-hydroxybutyrate, ethyl alcohol, methyl trichloride, zopiclone, or diethyl ether.

In one embodiment, the medicament is an agent for treating a wasting disease. In some embodiments, agents treating a wasting disease include, but are not limited to, corticosteroids, anabolic steroids, cannabinoids, metoclopramid, cisapride, medroxyprogesterone acetate, megestrol acetate, cyproheptadine, hydrazine sulfate, pentoxifylline, thalidomide, anticytokine antibodies, cytokine inhibitors, eicosapentaenoic acid, indomethacin, ibuprofen, melatonin, insulin, growth hormone, clenbuterol, porcine pancreas extract, IGF-1, IGF-1 analogue and secretagogue, myostatin analogue, proteasome inhibitor, testosterone, oxandrolone, enbrel, melanocortin 4 receptor agonist, or a combination thereof.

In one embodiment, the medicaments are anti-inflammatory agents. In one embodiment, the anti-inflammatory agent is a non-steroidal anti-inflammatory agent. In one embodiment, the non-steroidal anti-inflammatory agent is a cox-1 inhibitor. In one embodiment, the non-steroidal anti-inflammatory agent is a cox-2 inhibitor. In one embodiment, the non-steroidal anti-inflammatory agent is a cox-1 and cox-2 inhibitor. In some embodiments, non-steroidal anti-inflammatory agents include but are not limited to aspirin, salsalate, diflunisal, ibuprofen, fenoprofen, flubiprofen, fenamate, ketoprofen, nabumetone, piroxicam, naproxen, diclofenac, indomethacin, sulindac, tolmetin, etodolac, ketorolac, oxaprozin, or celecoxib. In one embodiment, the anti-inflammatory agent is a steroidal anti-inflammatory agent. In one embodiment, the steroidal anti-inflammatory agent is a corticosteroid.

In one embodiment, the route of administration may be parenteral, or a combination thereof. In another embodiment, the route may be intravenous, intra-arterial, transmucosal, intravascular, intraventricular, intracranial, inhalation (aerosol), nasal aspiration (spray), intranasal (drops), sublingual, oral, aerosol or suppository or a combination thereof. In one embodiment, the dosage regimen will be determined by skilled clinicians, based on factors such as exact nature of the condition being treated, the severity of the condition, the age and general physical condition of the patient, body weight, and response of the individual patient, etc. In one embodiment, the dosing regimen will comprise a series of applications of the formulation where a sustained response is achieved by providing for multiple exposures.

### Synthetic Peptides

The GtS polypeptide fragments of the present invention may be synthesized chemically using methods known in the art for synthesis of peptides and polypeptides. These methods generally rely on the known principles of peptide synthesis; most conveniently, the procedures can be performed according to the known principles of solid phase peptide synthesis.

As used herein “peptide” indicates a sequence of amino acids linked by peptide bonds. A polypeptide is generally a peptide of about 30 and more amino acids.

Polypeptide analogs and peptidomimetics are also included within the scope of the invention as well as salts and esters of the polypeptides of the invention are encompassed. A polypeptide analog according to the present invention may optionally comprise at least one non-natural amino acid and/or at least one blocking group at either the C terminus or N terminus. Salts of the peptides of the invention are physiologically acceptable organic and inorganic salts. The design of appropriate “analogs” may be computer assisted.

The term “polypeptidomimetic” means that a polypeptide according to the invention is modified in such a way that it includes at least one non-peptidic, bond such as, for example, urea bond, carbamate bond, sulfonamide bond, hydrazine bond, or any other covalent bond. The design of appropriate “polypeptidomimetic” may be computer assisted.

Salts and esters of the peptides of the invention are encompassed within the scope of the invention. Salts of the polypeptides of the invention are physiologically acceptable organic and inorganic salts. Functional derivatives of the polypeptides of the invention covers derivatives which may be prepared from the functional groups which occur as side chains on the residues or the N- or C-terminal groups, by means known in the art, and are included in the invention as long as they remain pharmaceutically acceptable, i.e., they do not destroy the activity of the polypeptide and do not confer toxic properties on compositions containing it. These derivatives may, for example, include aliphatic esters of the carboxyl groups, amides of the carboxyl groups produced by reaction with ammonia or with primary or secondary amines, N-acyl derivatives of free amino groups of the amino acid residues formed by reaction with acyl moieties (e.g., alkanoyl or carbocyclic aroyl groups) or O-acyl derivatives of free hydroxyl group (for example that of seryl or threonyl residues) formed by reaction with acyl moieties.

The term “amino acid” refers to compounds, which have an amino group and a carboxylic acid group, preferably in a 1,2-1,3-, or 1,4-substitution pattern on a carbon backbone. α-Amino acids are most preferred, and include the 20 natural amino acids (which are L-amino acids except for glycine) which are found in proteins, the corresponding D-amino acids, the corresponding N-methyl amino acids, side chain modified amino acids, the biosynthetically available amino acids which are not found in proteins (e.g., 4-hydroxy-proline, 5-hydroxy-lysine, citrulline, ornithine, canavanine, djenkolic acid, β-cyanolanine), and synthetically derived α-amino acids, such as amino-isobutyric acid, norleucine, norvaline, homocysteine and homoserine. β-Alanine and γ-amino butyric acid are examples of 1,3 and 1,4-amino acids, respectively, and many others are well known to the art. Statine-like isosteres (a dipeptide comprising two amino acids wherein the CONH linkage is replaced by a CHOH), hydroxyethylene isosteres (a dipeptide comprising two amino acids wherein the CONH linkage is replaced by a CHOHCH₂), reduced amide isosteres (a dipeptide comprising two amino acids wherein the CONH linkage is replaced by a CH₂NH linkage) and thioamide isosteres (a dipeptide comprising two amino acids wherein the CONH linkage is replaced by a CSNH linkage) are also useful residues for this invention.

The amino acids used in this invention are those, which are available commercially or are available by routine synthetic methods. Certain residues may require special methods for incorporation into the polypeptide, and sequential, divergent or convergent synthetic approaches to the peptide sequence are useful in this invention. Natural coded amino acids and their derivatives are represented by three-letter codes according to IUPAC conventions. When there is no indication, the L isomer was used.

Conservative substitutions of amino acids as known to those skilled in the art are within the scope of the present invention, as long as antigenicity is preserved in the substituted polypeptide. Conservative amino acid substitutions includes replacement of one amino acid with another having the same type of functional group or side chain e.g. aliphatic, aromatic, positively charged, negatively charged. These substitutions may enhance oral bioavailability, penetration into the central nervous system, targeting to specific cell populations and the like. One of skill will recognize that individual substitutions, deletions or additions to peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art.

The following six groups each contain amino acids that are conservative substitutions for one another:

### 1) Alanine (A), Serine (S), Threonine (T);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V); and

### 6) Phenylalanine (F), Tyrosine (Y), Tryptophan (W).

In one embodiment, the polypeptides of this invention include any polypeptide as herein described, any polypeptide which is homologous thereto, or in some embodiments, any polypeptide which specifically interacts with a polypeptide as herein described, which inhibits S. pneumoniae adhesion to and/or penetration of host cells.

In some embodiments, a polypeptide of this invention comprises an S. pneumoniae or fragment thereof, which is involved in bacterial adhesion and or invasion of a host cell, which in some embodiments is accomplished via interaction of a host cell protein or fragment, representing in some embodiments, a cognate binding pair, necessary for adhesion to and/or invasion of the host cell. In some embodiments, a polypeptide of this invention comprises a host cell protein or a fragment thereof, which interacts with S. pneumoniae, which is involved in bacterial adhesion and or invasion of the host cell. In some embodiments, a polypeptide of this invention interferes with the interaction, or in some embodiments, a polypeptide of this invention competes for binding to either member, or in some embodiments, both members of the cognate binding pair.

In some embodiments, this invention surprisingly has shown that S. pneumoniae adhesion specifically to epithelial cells of the respiratory system involves the GtS protein and that inhibition of this adhesion may be mediated by the isolated polypeptides as described herein, including polypeptides corresponding to the GtS protein specifically described, or fragments thereof, or in some embodiments, polypeptides homologous thereto. Such inhibition of adhesion may be particularly useful in treating sepsis mediated by S. pneumoniae. Moreover, the inhibition of adhesion by the polypeptides of this invention, in particular polypeptides corresponding to the GtS protein, or homologues thereof, or fragments thereof, was not species specific. In some embodiments, this invention unexpectedly provides for the treatment of infection, for example, and in some embodiments, treating latter stages of infection, or in some embodiments, early stages of infection, or in some embodiments, treating sepsis, by administering a GtS protein, or a homologue thereof, or a fragment thereof, derived from a first species of S. pneumoniae, whereas the subject may be infected with a different species of S. pneumonia.

In some embodiments of this invention, inhibition of S. pneumoniae adhesion specifically to epithelial cells of the respiratory system may be mediated by any isolated polypeptide which is described herein, or fragments or homologues thereof.

In one embodiment, polypeptides of the present invention are administered in combination. In one embodiment, treatments are administered to patients. In some embodiments, the GtS protein fragments of this invention and compositions comprising the same and vectors or polynucleotides encoding the same are administered to a subject infected with S. pneumoniae, wherein the subject has only recently become symptomatic, thereby being a treatment of early stages of infection in the subject. In some embodiments, the peptides, vectors, polynucleotides and compositions as herein described are administered during latter stages of infection, where fulminant pneumonia is present. In some embodiments, the peptides, vectors, polynucleotides and compositions as herein described are administered during latter stages of infection, when sepsis has occurred. The peptides, vectors, polynucleotides and compositions as herein described are administered during latter stages of infection, when multiple foci within a lung lobe are discerned, or in some embodiments, multiple lobes of the lung are affected. It is to be understood that the therapeutic compositions, peptides, vectors, polynucleotides and vaccines as herein described are administered at any point during infection with S. pneumoniae and are to be considered as part of this invention.

In some embodiments, administration of the compounds of this invention is intended to reduce the severity of a pathologic condition. By the term “reduce the severity of the pathologic condition”, it is to be understood that any reduction via the methods, compounds and compositions disclosed herein, is to be considered encompassed by the invention. Reduction in severity may, in one embodiment comprise enhancement of survival, or in another embodiment, halting disease progression, or in another embodiment, delay in disease progression or in another embodiment, amelioration of symptoms.

In some embodiments, administration of the compounds of this invention is intended to treat sepsis associated with S. pneumoniae infection. In one embodiment, treatments of this invention include the administration of a polypeptide, vector, nucleic acid, composition as herein described, administered to patients, whereby administration reduces the incidence, severity, or symptomatology associated with sepsis. In one embodiment, treatment of sepsis or latter stages of infection in a subject may be accomplished by the administration of any polypeptide of this invention, as herein described. In some embodiments, treatment of sepsis or latter stages of infection in a subject may be accomplished by the administration of a GtS polypeptide fragment as herein described, or any fragment thereof.

In some embodiments, the compositions of this invention will consist essentially of a polypeptide/polynucleotide/vector as herein described. In some embodiments, the term “consisting essentially of” refers to a composition whose only active ingredient of a particular class of agents, is the indicated active ingredient, however, other compounds may be included which are involved directly in the therapeutic effect of the indicated active ingredient. In some embodiments, the term “consisting essentially of” refers to a composition whose only active ingredient of targeting a particular mechanism, or acting via a particular pathway, is the indicated active ingredient, however, other compounds may be included which are involved directly in the therapeutic effect of the indicated active ingredient, which for example have a mechanism of action related to but not directly to that of the indicated agent. In some embodiments, the term “consisting essentially of” refers to a composition whose only active ingredient is the indicated active ingredient, however, other compounds may be included which are for stabilizing, preserving, etc. the formulation, but are not involved directly in the therapeutic effect of the indicated active ingredient. In some embodiments, the term “consisting essentially of” may refer to components which facilitate the release of the active ingredient. In some embodiments, the term “consisting” refers to a composition, which contains the active ingredient and a pharmaceutically acceptable carrier or excipient.

In one embodiment, “preventing, or treating” refers to any one or more of the following: delaying the onset of symptoms, reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, preventing relapse to a disease, decreasing the number or frequency of relapse episodes, increasing latency between symptomatic episodes, increasing time to sustained progression, expediting remission, inducing remission, augmenting remission, speeding recovery, or increasing efficacy of or decreasing resistance to alternative therapeutics. In one embodiment, “treating” refers to therapeutic treatment, wherein the object is to lessen the targeted pathologic condition or disorder as described hereinabove.

In another embodiment, “symptoms” may be any manifestation of a disease or pathological condition as described hereinabove.

In one embodiment, the subject for treatment is a mammal. In another embodiment the subject is human. In another embodiment, the subject is defined as elderly human. In one embodiment the definition of “elderly human” is a person over 65 years of age. In one embodiment, the subject for treatment is a child, whereby the definition of a “child” is a human under 4 years of age.

In one embodiment, the compositions described herein are aimed at treating sepsis caused by S. pneumonia infection in an individual. “Sepsis” is defined by the presence of bacteria (bacteremia) or other infectious organisms or their toxins in the blood (septicemia) or in other tissue of the body. Sepsis may be associated with clinical symptoms of systemic illness, such as fever, chills, malaise, low blood pressure, and mental status changes.

It is to be understood that any method of this invention may make use of the polypeptides as described herein, including fusion polypeptides comprising the GtS polypeptides fused to a targeting moiety and/or a second therapeutic protein. It is to be understood that any method of this invention may make use of any composition as described herein, including compositions, liposomes, etc., comprising a GtS polypeptide as described herein, and any form thereof, or a vector or nucleic acid comprising the same, for the treatment of S. pneumoniae infection, for the treatment of sepsis caused by S. pneumoniae infection, or for the prevention of S. pneumoniae adhesion to respiratory cells.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Example 1

### A GtS Fragment The amino acid sequence of S. pneumoniae GtS (accession code NP_{—}346492) is presented by SEQ ID NO:1:

| | |
|---|---|
| 1 | MSKDIRVRYA PSPTGLLHIG NARTALFNYL YARHHGGTFL IRIEDTDRKR HVEDGERSQL |
| 61 | ENLRWLGMDW DESPESHENY RQSERLDLYQ KYIDQLLAEG KAYKSYVTEE ELAAERERQE |
| 121 | VAGETPRYIN EYLGMSEEEK AAYIAEREAA GIIPTVRLAV NESGIYKWHD MVKGDIEFEG |
| 181 | GNIGGDWVIQ KKDGYPTYNF AVVIDDHDMQ ISHVIRGDDH IANTPKQLMV YEALGWEAPE |
| 241 | FGHMTLIINS ETGKKLSKRD TNTLQFIEDY RKKGYLPEAV FNFIALLGWN PGGEDEIFSR |
| 301 | EEFIKLFDEN RLSKSPAAFD QKKLDWMSND YIKNADLETI FEMAKPFLEE AGRLTDKAEK |
| 361 | LVELYKPQMK SVDEIIPLTD LFFSDFPELT EAEREVMTGE TVPTVLEAFK AKLEAMTDDE |
| 421 | FVTENIFPQI KAVQKETGIK GKNLFMPIRI AVSGEMHGPE LPDTIFLLGR EKSIQHIENM |
| 481 | LKEISK |

A fragment of the above protein lacking the N-terminal amino acids 1-332 amino acids was produces. The fragment denoted GtS(333-486), containing 154 amino acids corresponding to residues 333-486 of SEQ ID NO:1 is presented by SEQ ID NO:3:

MKNADLETIFEMAKPFLEEAGRLTDKAEKLVELYKPQMKSVDEIIPLT
DLFFSDFPELTEAEREVMTGETVPTVLEAFKAKLEAMTDDKFVTENIF
PQIKAVQKETGIKGKNLFMPIRIAVSGEMHGPELPDTIFLLGREKSIQ
HIENMLKEISK.

The nucleotides sequence of the fragment is presented in SEQ ID NO:11:

AAG AAT GCA GAC CTT GAA ACC ATC TTT GAA ATG GCA
AAA CCA TTC TTA GAG GAA GCA GGC CGT TTG ACT GAC
AAG GCT GAA AAA TTA GTT GAG CTC TAT AAA CCA CAA
ATG AAA TCA GTA GAT GAG ATT ATC CCA TTG ACA GAT
CTT TTC TTC TCA GAT TTC CCA GAA TTG ACA GAA GCA
GAG CGC GAA GTC ATG ACG GGT GAA ACA GTT CCA ACA
GTT CTT GAA GCA TTC AAA GCA AAA CTT GAA GCG ATG
ACA GAT GAT AAA TTT GTG ACA GAA AAT ATC TTC CCA
CAA ATT AAA GCA GTT CAA AAA GAA ACA GGT ATT AAA
GGG AAA AAT CTT TTC ATG CCT ATT CGT ATC GCA GTT
TCA GGC GAA ATG CAT GGG CCA GAA TTA CCA GAT ACA
ATT TTC TTG CTT GGA CGT GAA AAA TCA ATT CAG CAT
ATC GAA AAC ATG CTA AAA GAA ATC TCT AAA TAA.

### Example 2

### Homology to Human

A homology test comparing the GtS(333-486) fragment of SEQ ID NO:3 with the human genome sequences was performed using http://blast.ncbi.nlm.nih.gov/Blast.cgi.

The highest homology found was between the GtS fragment and the human protein glutamyl-tRNA synthetase 2 (Human GtS, GENE ID: 124454 EARS2). The overall gene identity (comparing SEQ ID NO:2 to Human GtS, GENE ID: 124454 EARS2) is 7.66%.

In contrast, the homology between the intact GtS protein sequence (SEQ ID NO:1) and the human GtS protein (Human GtS, GENE ID: 124454 EARS2) is 24%. Clearly, the GtS polypeptide fragment of SEQ ID NO:2 has significant less sequence identity to human proteins than the intact S. pneumoniae GtS protein.

### Example 2

### Homology to Different S. pneumoniae Strains

The NCBI-Blast tool, was used to check the homology between the GtS(333-486) fragment of SEQ ID NO:3 and other S. pneumoniae strains. As demonstrated in table 1, all S. pneumoniae strains tested have at least 98% identity to SEQ ID NO:3, and 100% identity to SEQ ID NO:1 (in the relevant regions).

TABLE 1

Sequence identity

| S. pneumoniae strain | to SEQ ID NO: 3 | to SEQ ID NO: 2 |
|---|---|---|
| SP14-BS69 | 100% | 100% |
| Hungary19A-6 | 100% | 100% |
| SP23-BS72 | 100% | 100% |
| SP6-BS73 | 100% | 100% |
| R6 | 100% | 100% |
| D39 | 100% | 100% |
| SP18-BS74 | 99% | 100% |
| G54 | 99% | 100% |
| TIGR4 | 99% | 100% |
| SP11-BS70 | 99% | 100% |
| MLV-016 | 99% | 100% |
| CDC1087-00 | 99% | 100% |
| SP19-BS75 | 99% | 100% |
| CDC0288-04 | 99% | 100% |
| CDC3059-06 | 98% | 100% |
| CGSP14 | 98% | 100% |
| SP195 | 98% | 100% |
| SP9-BS68 | 98% | 100% |
| SP3-BS71 | 98% | 100% |
| CDC1873-00 | 98% | 100% |

The sequence mutations founds between the strains (maximum two differences per each two strains) are: L/F 382, G/D 400, K/E 421, UV 466, and M/1481 (numbered according to SEQ ID NO:1).

### Example 3

### Cloning and Purification of the GtS Fragment

Cloning and purification of the GtS fragment were performed as described in Mizrachi-Nebenzahl et al. 2007, J Infect Dis. 196:945-53.

The GtS fragment was amplified from S. pneumoniae strain R6 genomic DNA by PCR with the following primers which contained Xoh1 and EcoRI recognition sequences, respectively:

Forward
(SEQ ID NO: 12)
5′GGAATTCAAGAATGCAGACCTTGAAACC 3′
Reverse
(SEQ ID NO: 13)
5′CCGCTCGAGTTATTTAGAGATTTCTTTTAGCAT 3′

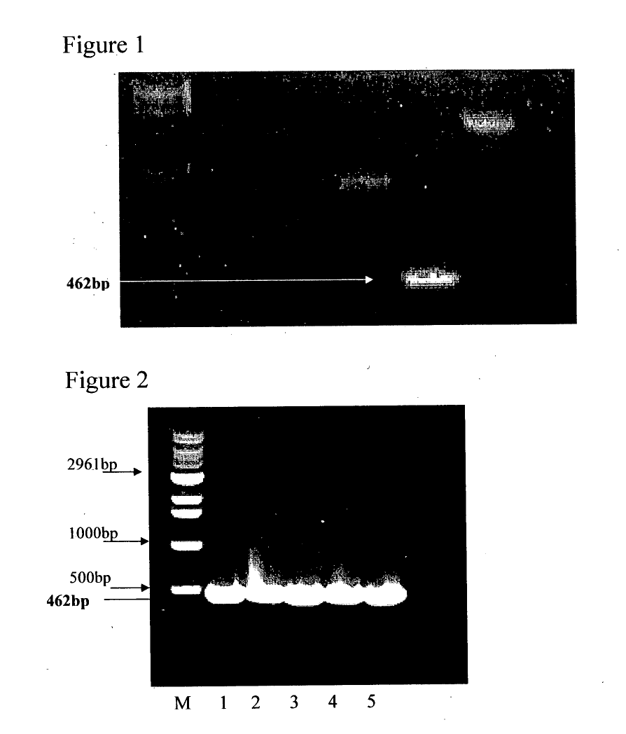

FIG. 1 represents amplification PCR of GtS(333-486) by genomic DNA.

The amplified and Xoh1-E.coRI (Takara Bio Inc, Shiga, Japan) digested DNA-fragments were cloned into the pET32a expression vector (BD Biosciences Clontech, Palo Alto, Calif., USA) and transformed in DH5a UltraMAX ultracompetent E. coli cells (Invitrogen, Carlsbad, Calif., USA). Ampicillin-resistant transformants were cultured and plasmid DNA was analyzed by PCR. The existence of the expected 462 bp size insert was confirmed by PCR amplification as shown in FIG. 2.

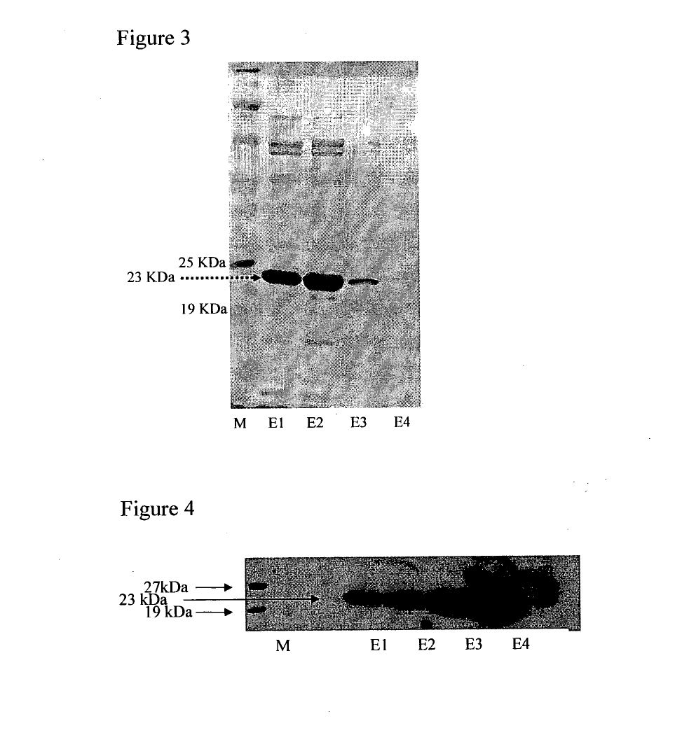

The modified (minus thioredoxin (TRX)) pET32a-GtS fragment vector was purified from DH5α UltraMAX cells using Qiagen High Speed Plasmid Maxi Kit (Qiagen GMBH, Hilden, Germany) and transformed in E. coli host expression strain BL21(DE3)pLysS (Stratagene, La Jolla, Calif.). The identity of the insert was confirmed by sequencing. Bacteria were grown over night and expression of the recombinant protein was induced by the addition of 1 mM IPTG to BL21(DE3)pLysS+6PGD cells for 5 hours. The cells were harvested by centrifugation, and lysed in lysis buffer. The HIS-tagged recombinant protein was purified using a Ni-NTA column (Qiagen GMBH, Hilden, Germany); binding for 1 hour at room temperature then the column was washed with wash buffer (8 M urea, 0.1 M NaH2PO₄, 0.01 M Tris-Cl pH 6.3), and the recombinant protein was recovered from the column using elution buffer (8 M urea, 0.1 M NaH₂PO₄, 0.01 M Tris-Cl, pH 5.9). Isolation of the protein was confirmed by Coomassie Brilliant blue staining and by Western blot analysis using anti-HIS antibodies (BD Biosciences Clontech, Palo Alto, Calif., USA). Resolution of the eluted protein by 1D-PAGE revealed a single band following staining with Coomassie Brilliant Blue (23 kDa band) as presented in FIG. 3. FIG. 4 represents western blot analysis 1D-PAGE using anti-HAT antibodies of the recombinant protein confirmed the 23k Da band to be HIS-tagged-rGtS(333-486) fusion protein.

### Example 4

### Inhibition of S. Pneumoniae Adhesion to Epithelial Cells Mediated by the rGtS Fragment

A549 cells (type II epithelial lung carcinoma cells) were cultured in the absence of antibiotics at a concentration of 2.5×10⁵ cells/well on average. P710 was added to the cells and incubated for 1 hour. Excess protein was removed and S. pneumoniae (10⁶CFU) were added for 1 hour. Excess bacteria were removed. Cultured cells were liberated and plated onto Blood agar plates.

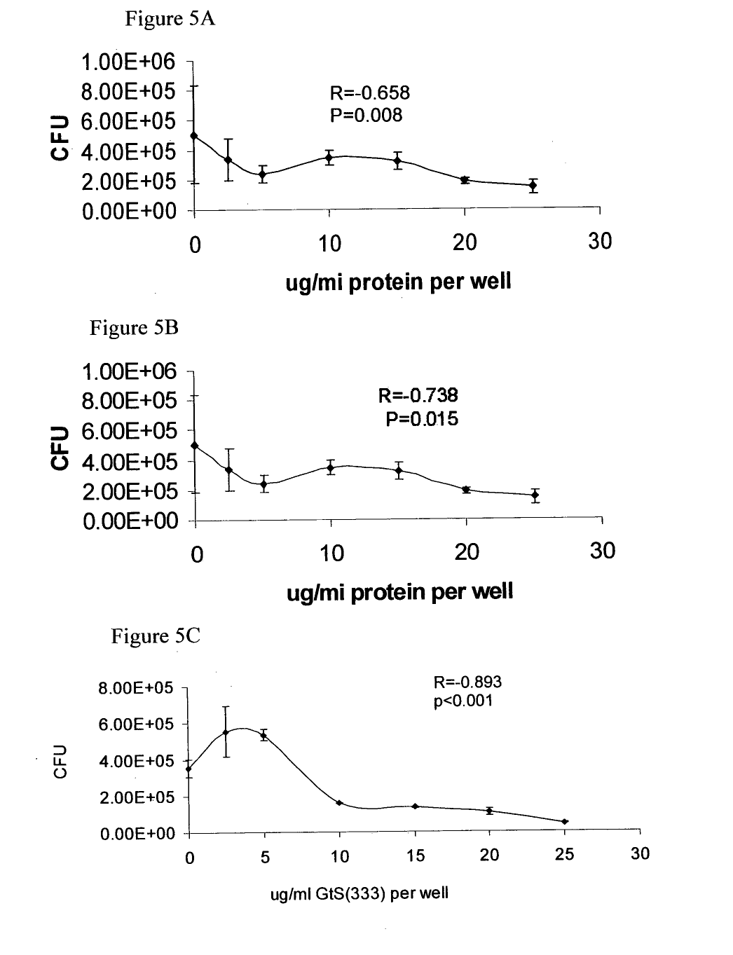

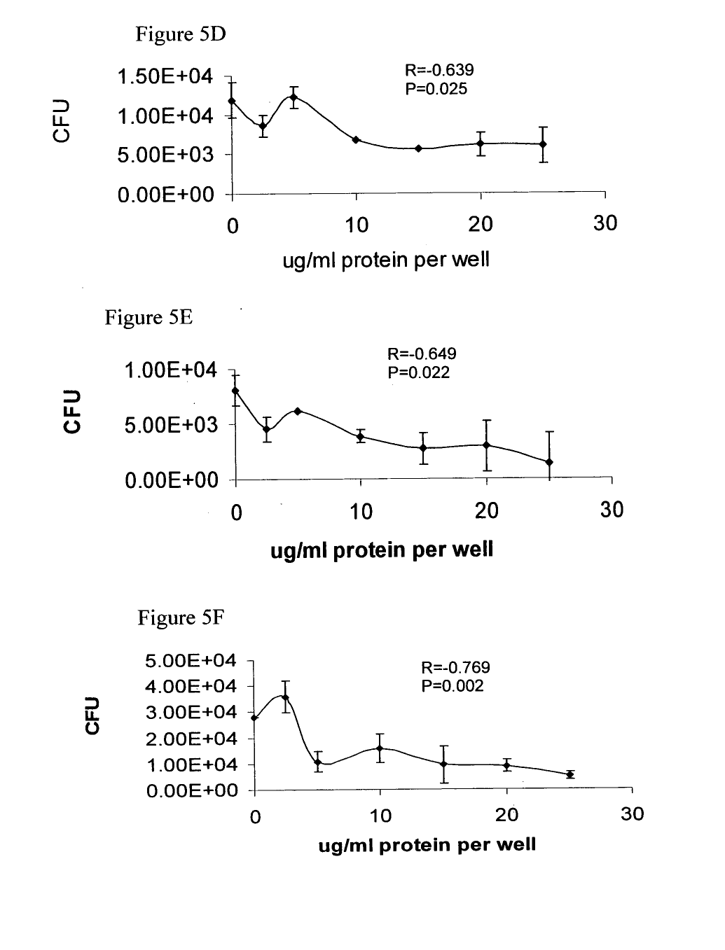

As can be seen in FIG. 5, every strain tested demonstrated a reduction in colony count numbers, indicating the cross-reactive nature of the peptide in eliciting inhibition of S. pneumoniae adhesion. The inhibition of unencapsulated serotype 2 strain R6 adhesion (panel A), serotype 2 strain D39 adhesion (panel B), unencapsulated serotype 3 strain 3.8 adhesion (panel C), serotype 3 strain WU2 adhesion (panel D), unencapsulated serotype 14 strain 14.8 adhesion (panel E) and serotype 14 strain 14 adhesion is shown.

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

1. A method of inhibiting adhesion of S. pneumoniae to cells of the respiratory tract, said method comprising contacting cells of a respiratory tract with a synthetic or recombinant polypeptide of 50-250 amino acids derived from the sequence of Streptococcus pneumonia (S. pneumoniae) glutamyl tRNA synthetase (GtS) of SEQ ID NO:1, comprising the sequence KNADLETDFEMAKPFLEEAGRLTDKAEKL (SEQ ID NO:2), and variants and analogs thereof, wherein said synthetic or recombinant polypeptide prevents adhesion of S. pneumoniae to cells of said respiratory tract.

2. The method of claim 1 wherein said synthetic or recombinant polypeptide consists of 100-200 amino acids.

3. The method of claim 1 wherein said synthetic or recombinant polypeptide shares less than 30% identity or homology with the human GtS protein sequence.

4. The method of claim 1 wherein said synthetic or recombinant polypeptide shares less than 10% identity or homology with the human GtS protein sequence.

5. The method of claim 1 wherein said synthetic or recombinant polypeptide comprises a sequence selected from the group consisting of:
XKNADLETIFEMAKPFLEEAGRLTDKAEKLVELYKPQMKSVDEIIPLTD LFFSDFPE LTEAEREVMTGETVPTVLEAFKAKLEAMTDDKFVTENIFPQIKAVQ KETGIKGKNLFMPIRIAVSGEMHGPELPDTIFLLGREKSIQHIENML KEISK (SEQ ID NO:3), wherein X is Methionine or represents the polypeptide's N-terminus, and variants and analogs thereof; and
XKNADLETIFEMAKPFLEEAGRLTDKAEKLVELYKPQMKSVDEIIPLTDX₁ FFSDFPELTEAEREVMTX₂ETVPTVLEAFKAKLEAMTDDX₃FVTENIFPQIKAVQKET GIKGKNLFMPIRIAVSGEMHGPELPDTX₄FLLGREKSIQHIENX₅L KEISK (SEQ ID NO:4 wherein X is Methionine or represents the polypeptide's N-terminus X is L or F, X₂ is G or D, X₃ is K or E, X₄ is I or V, and X₅ is M or I, and variants and analogs thereof.

6. (canceled)

7. The method of claim 1 wherein said synthetic or recombinant polypeptide comprises a sequence selected from the group consisting of SEQ ID NO:5-SEQ ID NO: 10:
XKNADLETIFEMAKPFLEEAGRLTDKAEKLVELYKP (SEQ ID NO:5);
MKNADLETIFEMAKPFLEEAGRLTDKAEKLVELYKPQMKSVDEIIPLTDLFFS DFP (SEQ ID NO:6);
XKNADLETIFEMAKPFLEEAG RLTDKAEKLVELYKPQMKSVDEIIPLTDLFFS DFPELTEAEREVMTGETVPTVLEAFKAK (SEQ ID NO:7);
MKNADLETIFEMAKPFLEEAG RLTDKAEKLVELYKPQMKSVDEIIPLTDLFFS DFPELTEAEREVMTGETVPTVLEAFKAKLEAMTDDKFVTENIFPQIKAVQKET (SEQ ID NO:8);
XKNADLETIFEMAKPFLEEAG RLTDKAEKLVELYKPQMKSVDEIIPLTDLFFS DFPELTEAEREVMTGETVPTVLEAFKAKLEAMTDDKFVTENIFPQIKAVQKE TGKGKNLFMPIRIAVSG (SEQ ID NO:9); and
XKNADLETIFEMAKPFLEEAG RLTDKAEKLVELYKPQMKSVDEIIP LTDLFFSDFPELTEAEREVMTGETVPTVLEAFKAKLEAMTDDKFVT ENIFPQIKAVQKETGIKGKNLFMPIRIAVSGEMHGPELPDTIFLLGR (SEQ ID NO: 10), wherein X is Methionine or represents the polypeptide's N-terminus, and variants and analogs thereof.

8. The method of claim 1 wherein said synthetic or recombinant polypeptide consists of a sequence selected from the group consisting of SEQ ID NO:3-SEQ ID NO: 10.

9. The method of claim 1 wherein said synthetic or recombinant polypeptide is conjugated or fused to a carrier protein.

10. The method of claim 1, further comprising the step of contacting cells of a respiratory tract with an isolated polynucleotide sequence encoding said recombinant polypeptide, wherein said polypeptide is expressed and is capable of contacting said respiratory tract.

11. The method of claim 1, comprising contacting cells of a respiratory tract with a composition comprising two or more synthetic or recombinant polypeptide.

12. (canceled)

13. A method of treating a subject infected with S. pneumoniae, said method comprising administering to said subject a synthetic or recombinant polypeptide of 50-250 amino acids derived from the sequence of Streptococcus pneumonia (S. pneumoniae) glutamyl tRNA synthetase (GtS) of SEQ ID NO:1, comprising the sequence KNADLETIFEMAKPFLEEAGRLTDKAEKL (SEQ ID NO:2), and variants and analogs thereof.

14. The method of claim 13 wherein said synthetic or recombinant polypeptide consists of 100-200 amino acids.

15. The method of claim 13 wherein said synthetic or recombinant polypeptide shares less than 30% identity or homology with the human GtS protein sequence.

16. The method of claim 13 wherein said synthetic or recombinant polypeptide shares less than 10% identity or homology with the human GtS protein sequence.

17. The method of claim 13 wherein said synthetic or recombinant polypeptide comprises a sequence selected from:
XKNADLETIFEMAKPFLEEAGRLTDKAEKLVELYKPQMKSVDEIIPLTDLFFSDFPE LTEAEREVMTGETVPTVLEAFKAKLEAMTDDKFVTENIFPQIKAVQ KETGIKGKNLFMPIRIAVSGEMHGPELPDTIFLLGREKSIQHIENML KEISK (SEQ ID NO:3), wherein X is Methionine or represents the polypeptide's N-terminus, and variants and analogs thereof; and XKNADLETIFEMAKPFLEEAGRLTDKAEKL VELYKPQMKSVDEIIPLTDX₁ FFSDFPELTEAEREVMTX₂ETVPTVLEAFKAKLEAMTDDX₃FVTENIFPQIK AVQKETGIKGKNLFMPIRIAVSGEMHGPELPDTX₄FLLGREKSIQHIENX₅L KEISK (SEQ ID NO:4), wherein X is Methionine or represents the polypeptide's N-terminus X₁ is L or F, X₂ is G or D, X₃ is K or E, X₄ is I or V, and X₅ is M or I, and variants and analogs thereof.

18. (canceled)

19. The method of claim 13 wherein said synthetic or recombinant polypeptide comprises a sequence selected from the group consisting of SEQ ID NO:5-SEQ ID NO: 10:
XKNADLETIFEMAKPFLEEAGRLTDKAEKLVELYKP (SEQ ID NO:5);
MKNADLETIFEMAKPFLEEAGRLTDKAEKLVELYKPQMKSVDEIIPLTDLFFS DFP (SEQ ID NO:6);
XKNADLETIFEMAKPFLEEAGRLTDKAEKLVELYKPQMKSVDEIIPLTDLFFS DFPELTEAEREVMTGETVPTVLEAFKAK (SEQ ID NO:7);
MKNADLETIFEMAKPFLEEAGRLTDKAEKLVELYKPQMKSVDEIIPLTDLFFS DFPELTEAEREVMTGETVPTVLEAFKAKLEAMTDDKFVTENIFPQIKAVQKE T (SEQ ID NO:8);
XKNADLETIFEMAKPFLEEAGRLTDKAEKLVELYKPQMKSVDEIIPLTDLFFS DFPELTEAEREVMTGETVPTVLEAFKAKLEAMTDDKFVTENIFPQIKAVQKE TGIKGKNLFMPIRIAVSG (SEQ ID NO:9); and
XKNADLETIFEMAKPFLEEAGRLTDKAEKLVELYKPQMKSVDEIIP LTDLFFSDFPELTEAEREVMTGETVPTVLEAFKAKLEAMTDDKFVT ENIFPQIKAVQKETGIKGKNLFMPIRIAVSGEMHGPELPDTIFLLGR (SEQ ID NO:10), wherein X is Methionine or represents the polypeptide's N-terminus, and variants and analogs thereof.

20. The method of claim 13 wherein said synthetic or recombinant polypeptide consists of a sequence selected from the group consisting of SEQ ID NO:3-SEQ ID NO: 10.

21. The method of claim 13 wherein said synthetic or recombinant polypeptide is conjugated or fused to a carrier protein.

22. The method of claim 13, further comprising the step of administering an isolated polynucleotide sequence encoding said recombinant polypeptide to said subject, wherein said polypeptide is capable of being expressed in said subject.

23. The method of claim 13, further comprising administering a composition comprising two or more synthetic or recombinant polypeptide to said subject.

24. (canceled)

25. The method of claim 13, wherein said subject is afflicted with sepsis.

26-49. (canceled)


SEQUENCE LISTING (15 sequences)

SEQ ID NO: 1 - protein, 486 aa, Streptococcus pneumoniae
MSKDIRVRYAPSPTGLLHIGNARTALFNYLYARHHGGTFLIRIEDTDRKRHVEDGERSQLENLRWLGMDWDESPESHENYRQSERLDLYQKYIDQLLAEGKAYKSYVTEEELAAERERQEVAGETPRYINEYLGMSEEEKAAYIAEREAAGIIPTVRLAVNESGIYKWHDMVKGDIEFEGGNIGGDWVIQKKDGYPTYNFAVVIDDHDMQISHVIRGDDHIANTPKQLMVYEALGWEAPEFGHMTLIINSETGKKLSKRDTNTLQFIEDYRKKGYLPEAVFNFIALLGWNPGGEDEIFSREEFIKLFDENRLSKSPAAFDQKKLDWMSNDYIKNADLETIFEMAKPFLEEAGRLTDKAEKLVELYKPQMKSVDEIIPLTDLFFSDFPELTEAEREVMTGETVPTVLEAFKAKLEAMTDDEFVTENIFPQIKAVQKETGIKGKNLFMPIRIAVSGEMHGPELPDTIFLLGREKSIQHIENMLKEISK

SEQ ID NO: 2 - protein, 29 aa, Artificial Sequence
  Synthetic
KNADLETIFEMAKPFLEEAGRLTDKAEKL

SEQ ID NO: 3 - protein, 155 aa, Artificial Sequence
  Synthetic
XKNADLETIFEMAKPFLEEAGRLTDKAEKLVELYKPQMKSVDEIIPLTDLFFSDFPELTEAEREVMTGETVPTVLEAFKAKLEAMTDDKFVTENIFPQIKAVQKETGIKGKNLFMPIRIAVSGEMHGPELPDTIFLLGREKSIQHIENMLKEISK

SEQ ID NO: 4 - protein, 155 aa, Artificial Sequence
  Synthetic
XKNADLETIFEMAKPFLEEAGRLTDKAEKLVELYKPQMKSVDEIIPLTDXFFSDFPELTEAEREVMTXETVPTVLEAFKAKLEAMTDDXFVTENIFPQIKAVQKETGIKGKNLFMPIRIAVSGEMHGPELPDTXFLLGREKSIQHIENXLKEISK

SEQ ID NO: 5 - protein, 36 aa, Artificial Sequence
  Synthetic
XKNADLETIFEMAKPFLEEAGRLTDKAEKLVELYKP

SEQ ID NO: 6 - protein, 55 aa, Artificial Sequence
  Synthetic
KNADLETIFEMAKPFLEEAGRLTDKAEKLVELYKPQMKSVDEIIPLTDLFFSDFP

SEQ ID NO: 7 - protein, 81 aa, Artificial Sequence
  Synthetic
XKNADLETIFEMAKPFLEEAGRLTDKAEKLVELYKPQMKSVDEIIPLTDLFFSDFPELTEAEREVMTGETVPTVLEAFKAK

SEQ ID NO: 8 - protein, 106 aa, Artificial Sequence
  Synthetic
XKNADLETIFEMAKPFLEEAGRLTDKAEKLVELYKPQMKSVDEIIPLTDLFFSDFPELTEAEREVMTGETVPTVLEAFKAKLEAMTDDKFVTENIFPQIKAVQKET

SEQ ID NO: 9 - protein, 123 aa, Artificial Sequence
  Synthetic
XKNADLETIFEMAKPFLEEAGRLTDKAEKLVELYKPQMKSVDEIIPLTDLFFSDFPELTEAEREVMTGETVPTVLEAFKAKLEAMTDDKFVTENIFPQIKAVQKETGIKGKNLFMPIRIAVSG

SEQ ID NO: 10 - protein, 139 aa, Artificial Sequence
  Synthetic
XKNADLETIFEMAKPFLEEAGRLTDKAEKLVELYKPQMKSVDEIIPLTDLFFSDFPELTEAEREVMTGETVPTVLEAFKAKLEAMTDDKFVTENIFPQIKAVQKETGIKGKNLFMPIRIAVSGEMHGPELPDTIFLLGR

SEQ ID NO: 11 - DNA, 465 bp, Artificial Sequence
  Recombinant
aagaatgcagaccttgaaaccatctttgaaatggcaaaaccattcttagaggaagcaggccgtttgactgacaaggctgaaaaattagttgagctctataaaccacaaatgaaatcagtagatgagattatcccattgacagatcttttcttctcagatttcccagaattgacagaagcagagcgcgaagtcatgacgggtgaaacagttccaacagttcttgaagcattcaaagcaaaacttgaagcgatgacagatgataaatttgtgacagaaaatatcttcccacaaattaaagcagttcaaaaagaaacaggtattaaagggaaaaatcttttcatgcctattcgtatcgcagtttcaggcgaaatgcatgggccagaattaccagatacaattttcttgcttggacgtgaaaaatcaattcagcatatcgaaaacatgctaaaagaaatctctaaataa

SEQ ID NO: 12 - protein, 523 aa, Homo sapiens
MAALLRRLLQRERPSAASGRPVGRREANLGTDAGVAVRVRFAPSPTGFLHLGGLRTALYNYIFAKKYQGSFILRLEDTDQTRVVPGAAENIEDMLEWAGIPPDESPRRGGPAGPYQQSQRLELYAQATEALLKTGAAYPCFCSPQRLELLKKEALRNHQTPRYDNRCRNMSQEQVAQKLAKDPKPAIRFRLEQVVPAFQDLVYGWNRHEVASVEGDPVIMKSDGFPTYHLACVVDDHHMGISHVLRGSEWLVSTAKHLLLYQALGWQPPHFAHLPLLLNRDGSKLSKRQGDVFLEHFAADGFLPDSLLDIITNCGSGFAENQMGRTLPELITQFNLTQVTCHSALLDLEKLPEFNRLHLQRLVSNESQRRQLVGKLQVLVEEAFGCQLQNRDVLNPVYVERILLLRQGHICRLQDLVSPVYSYLWTRPAVGRAQLDAISEKVDVIAKRVLGLLERSSMSLTQDMLNGELKKLSEGLEGTKYSNVMKLLRMALSGQQQGPPVAEMMLALGPKEVRERIQKVVSS

SEQ ID NO: 13 - DNA, 28 bp, Artificial Sequence
  Primer
ggaattcaagaatgcagaccttgaaacc

SEQ ID NO: 14 - DNA, 33 bp, Artificial Sequence
  Primer
ccgctcgagttatttagagatttcttttagcat

SEQ ID NO: 15 - DNA, 468 bp, Artificial Sequence
  Artificial recombinant
atgaaaaacgctgatctggaaactatttttgaaatggcaaaaccgtttctggaagaagcaggtcgtctgactgacaaagcagagaaactggttgagctgtacaaaccgcagatgaaatctgttgacgagatcattccgctgactgacctgttcttttctgatttcccggaactgactgaagcagaacgtgaagtaatgactggtgaaactgttccgactgttctggaagcgttcaaagctaaactggaggctatgaccgacgataaattcgtcaccgaaaacatctttccgcagatcaaagcggttcagaaagaaaccggtatcaaaggcaaaaacctgttcatgccgattcgtattgcagtatctggtgaaatgcatggtccggaactgccggatactatctttctgctgggtcgtgagaaatctatccagcacattgagaacatgctgaaagagatctccaaataa